(19) United States
(12) Patent Application Publication
(10) Pub. No.: US 2012/0328588 A1
(43) Pub. Date: Dec. 27, 2012

(54) COMPOUND PREPARATION AND ITS USES FOR PREVENTION AND TREATMENT OF HEARING IMPAIRMENT

(75) Inventors: Shen Gao, Shanghai (CN); Jing Gao, Shanghai (CN); Quangang Zhu, Shanghai (CN); Xiying Wang, Shanghai (CN); Xueying Ding, Shanghai (CN); Wei Zhang, Shanghai (CN); Xiaoyu Wang, Shanghai (CN); Min Zhang, Shanghai (CN); Xin Wu, Shanghai (CN); Lihua Ye, Shanghai (CN)

(73) Assignee: The Second Military Medical University of Chinese PLA, Shanghai (CN)

(21) Appl. No.: 13/582,755

(22) PCT Filed: May 8, 2011
(86) PCT No.: PCT/CN2011/073796

§ 371 Date: Sep. 5, 2012

(30) Foreign Application Priority Data
May 24, 2010 (CN) 201010180892.X

(51) Int. Cl. A61K 31/4422; A61P 27/16

(52) U.S. Cl. 424/94.1; 514/440

(57) ABSTRACT

A compound preparation contains alpha-lipoic acid and nimodipine at a ratio from 5:1 to 40:1. The above-mentioned compound preparation can be used for the prevention and treatment of noise-induced hearing impairment. This compound preparation not only increases the efficacy but decreases the dosage of nimodipine, reduces the side effects and improves the patient compliance.

### FIELD OF THE INVENTION

The present invention relates to the medical technology and especially relates to a compound preparation that is used for the prevention and treatment of noise-induced hearing impairment.

### BACKGROUND OF THE INVENTION

Alpha-lipoic acid (alpha 1ipoic acid, ALA), whose chemical name is 1-2 dithiolane-3-pentanoic acid, is a kind of natural disulfide, which was first isolated from the pig liver, and belongs to a kind of B-vitamin. Alpha-lipoic acid is a high effective and lipophilic radical scavenger which could be dissolved in the aqueous phase and lipid phase, easily cross through membranes and penetrate the blood brain barrier. It can be used for the prevention and treatment of diseases associated with free radicals, such as cancer, aging, diabetes, atherosclerosis, and degenerative diseases and disorders of brain and nerve tissues.

Nimodipine (NIM) is a kind of Ca²⁺ channel blockers, and can be used to relieve vasospasm by effectively preventing Ca²⁺ from entering cells and thus inhibiting smooth muscle contraction. Nimodipine is often used for the clinical treatment of cerebral vasospasm, cerebral infarction, cerebral arteriosclerosis and senile brain dysfunction.

Noise is a common environmental factor affecting human health. Persistent noise stimulation can affect cochlear hair cells by increasing their demand on ATP, oxygen and glucose that are needed to support cells, producing local ischemia and resulting in hair cell and spiral organ degeneration. Other studies found that superoxide anionic radical appeared in the cochlea spiral stria vascularis and the hydroxyl radical level significantly rose after noise exposure, indicating that excessively production of free radical is an important cause contributing to noise-induced hearing impairment. The increased content of radicals and overload of intracellular calcium induce abnormal change in cell structure, static cilia, DNA and protein, eventually leading to cell necrosis and apoptosis.

The influence of noise on hearing function mainly present as a decline in sensitivity of auditory sense, a rise in hearing threshold, and deterioration of language acceptance and signal discrimination, causing deafness in serious cases. Noise-induced hearing threshold shift can be temporary or permanent. Firstly, auditory sense adaptation and temporary hearing threshold shift appear, but these changes are usually physiological and can be recovered. Persistent contacts with high noise may cause permanent hearing threshold shift. Prolonged contacts can cause all frequency damage or even lead to noise-induced hearing loss, which is usually irreversible. Besides hearing impairment, noise can also cause many adverse effects, such as headache, dizziness, tinnitus, insomnia and muscle weakness.

Drugs commonly used for the prevention and treatment of hearing impairment mainly include drugs of improving microcirculation, such as: carbogen (mixed gas including 95% oxygen and 5% carbon dioxide), calcium antagonists, corticosteroids, ATP, denitration diop sodium, low molecular dextran, gingko leaf preparations, rhizoma ligustici wallichii, radix salviae miltiorrhizae and puerarin; drugs of promoting neurotrophic metabolism, including neurotrophic factor, vitamin B1 and vitamin B12; and drugs of scavenging oxygen free radicals, including oxygen free radical scavenging agents, antioxidant SOD, vitamin C and vitamin E. Despite the increasing number of patients with noise-induced hearing impairment, there is no drug available for effective and selective treatment of noise-induced hearing impairment.

### BRIEF SUMMARY OF THE INVENTION

It is one object of the present invention to provide a compound preparation that is used to effectively treat diseases caused by increased free radicals and Ca²⁺.

In addition, the present invention also provides a compound preparation for the treatment of noise-induced hearing impairment.

To solve the technical problem, the present invention was conducted as follows:

In one aspect, the present invention provides a compound preparation containing alpha-lipoic acid and nimodipine, and the ratio of alpha-lipoic acid to nimodipine content is from 5:1 to 40:1. The alpha-lipoic acid contains levorotatory body and/or dextral body. The preferable preparation is that in each unit of the preparation from 200 mg to 1000 mg includes alpha-lipoic acid from 40 mg to 400 mg, and nimodipine from 1 mg to 80 mg. The more preferable preparation is that in each unit of the preparation from 200 mg to 1000 mg includes alpha-lipoic acid from 80 mg to 200 mg, and nimodipine from 2 mg to 40 mg.

The dosage form of the compound preparation contains a solid tablet, an orally disintegrating formulation, and a granular formulation or a capsule.

In another aspect, the present invention provides use of the compound preparation for preventing noise-induced hearing impairment. The optimal ratio of the content of alpha-lipoic acid to that of nimodipine is 20:1 when the compound preparation is used to prevent noise-induced hearing impairment.

In the other aspect, the present invention provides use of compound preparation for treating noise-induced hearing impairment. The optimal ratio of alpha-lipoic acid to nimodipine content is 10:1 when the compound preparation is used to treat noise-induced hearing impairment.

This invented compound preparation utilizes the synergistic effect between the alpha-lipoic acid (which can scavenge xygen free radicals) and nimodipine (which is a Ca²⁺ antagonist). This compound preparation not only increases the efficacy but decreases the dosage of nimodipine, reduces the adverse effects, and improves patient compliance. Animal experiments showed that the invented compound preparation has a good pharmacodynamic synergy and can be used to prepare the medicine for the prevention and treatment of noise-induced hearing impairment.

### DETAILED DESCRIPTION OF THE INVENTION

The invention presents a compound preparation containing alpha-lipoic acid and nimodipine. The ratio of alpha-lipoic acid to nimodipine content is from 5:1 to 40:1. This invented compound preparation utilizes the synergistic effect between the alpha-lipoic acid (which can scavenge xygen free radicals) and nimodipine (which is a Ca²⁺ antagonist). It has a collaborative treatment for hearing impairment at two aetiological aspects in an adapted dosage.

The preferable preparation is that in each unit of the preparation from 200 mg to 1000 mg includes alpha-lipoic acid from 40 mg to 400 mg, and nimodipine from 1 mg to 80 mg. The more preferable preparation is that in each unit of the preparation from 200 mg to 1000 mg includes alpha-lipoic acid from 80 mg to 200 mg, and nimodipine from 2 mg to 40 mg.

### Example 1

### Preventive Effect of the Compound Preparations on Noise-Induced Hearing Impairment

Experiments with Hearing Threshold Shift Sixty-four guinea pigs with normal weight and pure white hair were randomly divided into control group, alpha-lipoic acid group, nimodipine group, and compound preparation group. The guinea pigs were exposed to an octave band of noise (OBN) centered at 4 kHz, 115 dB SPL, 4 h per day. Two days before noise exposure, animals in the study groups were administered with the corresponding drug dose as designed, and those in the control group were administered with normal saline (NS). Hearing threshold shift of the animals was determined at different intervals of noise exposure in different groups. The synergistic effect of alpha-lipoic acid and nimodipine on regulating hearing threshold shift was observed and analyzed using q test. The experimental results were in the following table 1 and table 2.

TABLE 1
Hearing threshold shift of the guinea pigs after noise exposure

| | | Mean change in hearing | |
|---|---|---|---|
| | | threshold shift (dB) | |
| | | At 1^{st} after | At 7^{th} day after |
| Group | Dose (mg/kg) | exposure | exposure |
| control | 0 | 39.6 | 22.5 |
| LA | 80 | 32.5 | 20.5 |
| NIM | 2 | 41.0 | 23.5 |
| NIM | 4 | 38.5 | 24 |
| NIM | 8 | 38 | 22.0 |
| NIM | 16 | 36 | 22.0 |
| LA + NIM | 80 + 2 | 32.0 | 21.0 |
| LA + NIM | 80 + 4 | 28* | 15.0* |
| LA + NIM | 80 + 8 | 27.0* | 17.5* |
| LA + NIM | 80 + 16 | 27.0* | 16.5* |

In table 1,
LA indicates alpha-lipoic acid, and NIM represents nimodipine.
Mean change in hearing threshold shift corresponding to change in hearing threshold shift before and afte noise exposure.
Compared with control, *P < 0.05, **P < 0.01.

TABLE 2
The synergistic effect of the alpha-lipoic acid and nimodipine.

| Group | Dose (mg/kg) | P | q |
|---|---|---|---|
| LA | 80 | Pla = 2/8 | 0.86 |
| NIM | 2 | Pnim = 2/8 | |
| LA + NIM | 80 + 2 | Pla + nim = 3/8 | |
| LA | 80 | Pla = 2/8 | 1.41 |
| NIM | 4 | Pnim = 3/8 | |
| LA + NIM | 80 + 4 | Pla + nim = 6/8 | |
| LA | 80 | Pla = 2/8 | 1.04 |
| NIM | 8 | Pnim = 5/8 | |
| LA + NIM | 80 + 8 | Pla + nim = 6/8 | |
| LA | 80 | Pla = 2/8 | 1.08 |
| NIM | 16 | Pnim = 5/8 | |
| LA + NIM | 80 + 16 | Pla + nim = 6/8 | |

Table 2 shows the mean hearing threshold shift within 7 days of exposure to noise. Being less than 20 dB was considered as an effective value. The synergistic effect of alpha-lipoic acid and nimodipine was analyzed using q test. Pla=2/8 means that there were 8 guinea pigs in the alpha-lipoic acid group, within which 2 were effective, and the remaining 6 were invalid. The mean of the rest was analogous to Pla=2/8.

The results indicate that when the ratio of alpha-lipoic acid to nimodipine was from 5:1 to 40:1, the compound preparation had a synergistic preventive effect on hearing threshold shift of the guinea pigs exposed to noise. The synergistic preventive effect was most significant when the ratio of alpha-lipoic acid to nimodipine was 20:1.

### Example 2

### Therapeutic Effect of the Compound Preparations on Noise-Induced Hearing Impairment

Experiments with Hearing Threshold Shift

Eighty guinea pigs with normal weight and pure white hair were randomly divided into control group, alpha-lipoic acid group, nimodipine group, and compound preparation group. The animals were exposed to an octave band of noise (OBN) centered at 4 kHz, 115 dB SPL, 4 h per day. Animals in the study groups were administered intravenously with the corresponding drug dose as designed, and those in the control group were administered with NS within 7 days after noise exposure. Hearing threshold shift of the animals was determined at different intervals of noise exposure in different groups. The synergistic effect of alpha-lipoic acid and nimodipine on regulating hearing threshold shift was observed and analyzed using q test. The experimental results were in the following table 3 and table 4.

TABLE 3
Hearing threshold shift of the guinea pigs after noise exposure

| | | Mean change in hearing | |
|---|---|---|---|
| | | threshold shift (dB) | |
| | Dose | At 1^{st} day after | 2 weeks after |
| Group | (mg/kg) | exposure | exposure |
| control | 0 | 39.6 | 22.0 |
| LA | 80 | 32.5 | 20.5 |
| NIM | 2 | 41.0 | 23.5 |
| NIM | 4 | 38.5 | 24 |
| NIM | 8 | 38 | 22.0 |
| NIM | 16 | 36 | 22.0 |
| LA + NIM | 80 + 2 | 32.0 | 20.0 |
| LA + NIM | 80 + 4 | 28.5* | 20.0 |
| LA + NIM | 80 + 8 | 27.0* | 14.0* |
| LA + NIM | 80 + 16 | 27.0* | 14.0* |

In table 3,
LA indicates alpha-lipoic acid, and NIM represents nimodipine.
Mean change in hearing threshold shift corresponding to change in hearing threshold shift before and after noise exposure.
Compared with control, *P < 0.05, **P < 0.01.

TABLE 4
The synergistic effect of alpha-lipoic acid and nimodipine.

| Group | Dose (mg/kg) | P | q |
|---|---|---|---|
| LA | 80 | Pla = 4/10 | 1.30 |
| NIM | 2 | Pnim = 1/10 | |
| LA + NIM | 80 + 2 | Pla + nim = 6/10 | |
| LA | 80 | Pla = 4/10 | 1.55 |
| NIM | 4 | Pnim = 3/10 | |
| LA + NIM | 80 + 4 | Pla + nim = 7/10 | |
| LA | 80 | Pla = 4/10 | 1.59 |
| NIM | 8 | Pnim = 3/10 | |
| LA + NIM | 80 + 8 | Pla + nim = 9/10 | |
| LA | 80 | Pla = 4/10 | 1.59 |
| NIM | 16 | Pnim = 3/10 | |
| LA + NIM | 80 + 16 | Pla + nim = 9/10 | |

Table 2 shows the mean hearing threshold shift within 7 days after noise exposure. Being less than 20 dB was considered as an effective value. The synergistic effect of alpha-lipoic acid and nimodipine was analyzed using q test. Pla=4/10 means that there were 10 guinea pigs in the alpha-lipoic acid group, within which 4 were effective, and the remaining 6 were invalid. The mean of the rest was analogous to Pla=4/10.

The results indicate that when the ratio of alpha-lipoic acid to nimodipine was from 5:1 to 40:1, the compound preparation had a synergistic treatment effect on hearing threshold shift of the guinea pigs exposed to noise. The synergistic treatment effect was most significant when the ratio of alpha-lipoic acid to nimodipine was 10:1.

### Example 3

The preparation (200˜1000 mg dosage): alpha-lipoic acid vs nimodipine=5:1, the adequate amount of disintegrants, fillers, adhesives, glidants, lubricants. The drugs and the excipients were made to an orally disintegrating formulation or a granular formulation.

Preparation method: nimodipine and excipients were dried for 2 h at 40° C., and alpha-lipoic acid was dried for 2 h at room temperature under vacuum conditions. The drugs and the excipients were respectively sieved through 180 μm pore size, precisely weighed, and mixed for 30 min using equal quantity gradual-increasing method. The mixture was weighed and made to an orally disintegrating formulation or a granular formulation.

### Example 4

The preparation (200˜1000 mg dosage): alpha-lipoic acid vs nimodipine=10:1, the adequate amount of disintegrants, fillers, adhesives, glidants, lubricants. The drugs and the excipients were made to capsules.

Preparation method: nimodipine and excipients were dried for 2 h at 40° C., and alpha-lipoic acid was dried for 2 h at room temperature under vacuum conditions. The drugs and the excipients were respectively sieved through 180 μm pore size, precisely weighed, and mixed for 30 min using equal quantity gradual-increasing method. The mixture was weighed and made to capsules.

### Example 5

The preparation (200˜1000 mg dosage): alpha-lipoic acid vs nimodipine=20:1, the adequate amount of disintegrants, fillers, adhesives, glidants, lubricants. The drugs and the excipients were made to an orally disintegrating formulation or a granular formulation.

Preparation method: nimodipine and excipients were dried for 2 h at 40° C., and alpha-lipoic acid was dried for 2 h at room temperature under vacuum conditions. The drugs and the excipients were respectively sieved through 180 μm pore size, precisely weighed, and mixed for 30 min using equal quantity gradual-increasing method. The mixture was weighed and made to an orally disintegrating formulation or a granular formulation.

### Example 6

The preparation (200˜1000 mg dosage): alpha-lipoic acid vs nimodipine=40:1, the adequate amount of disintegrants, fillers, adhesives, glidants, lubricants. The drugs and the excipients were made to tablets.

Preparation method: nimodipine and excipients were dried for 2 h at 40° C., and alpha-lipoic acid was dried for 2 h at room temperature under vacuum conditions. The drugs and the excipients were respectively sieved through 180 μm pore size, precisely weighed, and mixed for 30 min using equal quantity gradual-increasing method. The mixture was weighed and compressed to tablets.

While some of the embodiments of the present invention have been described in detail previously, it is still possible for those of ordinary skill in the art to make various modifications and changes to the particular embodiments shown without substantially departing from the teaching and advantages of the present invention. Such modifications and changes are encompassed in the spirit and scope of the present invention as set forth in the appended claims.

The claims defining the invention are as follows:

1. A compound preparation which contains alpha-lipoic acid and nimodipine, and the ratio of alpha-lipoic acid to nimodipine content is from 5:1 to 40:1.

2. The compound preparation of claim 1 wherein the alpha-lipoic acid contains levorotatory body and/or dextral body.

3. The compound preparation of claim 1 wherein each unit of the preparation from 200 mg to 1000 mg includes alpha-lipoic acid from 40 mg to 400 mg, and nimodipine from 1 mg to 80 mg.

4. The compound preparation of claim 3 wherein each unit of the preparation from 200 mg to 1000 mg includes alpha-lipoic acid from 80 mg to 200 mg, and nimodipine from 2 mg to 40 mg.

5. The compound preparation of claim 1 wherein the dosage form of the compound preparation contains a solid tablet, an orally-disintegrating formulation, a granular formulation or a capsule.

6. Use of compound preparation as claimed in claim 1, for preventing noise-induced hearing impairment.

7. The use of compound preparation as claimed in claim 6 wherein the ratio of alpha-lipoic acid to nimodipine content is 20:1.

8. Use of compound preparation as claimed in claim 1, for treating noise-induced hearing impairment.

9. The use of compound preparation as claimed in claim 8 wherein the ratio of alpha-lipoic acid to nimodipine content is 10:1.
